(19) United States
(12) Patent Application Publication
(10) Pub. No.: US 2008/0262618 A1
(43) Pub. Date: Oct. 23, 2008

(54) DEVICE FOR CARTILAGE REPAIR

(75) Inventors: Egidius Gerardus Maria Hermsen, Eindhoven (NL); Hermannus Hendricus Weinans, Driebergen-Rijsenburg (NL); Ronnij Matthieu Versteegen, Hegelsom (NL); Eva Wisse, Eindhoven (NL); Egbert Willem Meijer, Waalre (NL)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: JOINTSPHERE B.V., Eindhoven (NL)

(21) Appl. No.: 12/108,044

(22) Filed: Apr. 23, 2008

(30) Foreign Application Priority Data
Apr. 23, 2007 (EP) 07106748.2

(51) Int. Cl. A61F 2/30; A61B 17/08

(52) U.S. Cl. 623/14.12; 606/151

(57) ABSTRACT

A prosthesis device comprising a body at least partly formed from a biocompatible segmented thermoplastic elastomer having crystallized blocks, and at least one functional component which is able to reversibly bond to the crystallized blocks, wherein the elastomer has cartilage regenerative properties. A method is provided for the preparation of the biocompatible elastomer having cartilage regenerative properties, and a method for incorporating the biocompatible elastomer in a prosthesis device able to grow into cartilage.

### PRIORITY CLAIM

This patent application claims priority to European Patent Application No. 07106748.2, filed Apr. 23, 2007, the disclosure of which is incorporated herein by reference in its entirety.

### FIELD

The present disclosure relates to a prosthetic device for use in the joint space between two or more bones, more preferably in the joint space between the femoral condyle and the tibial plateau and/or for use in a bone structure. The present disclosure also relates to a biocompatible elastomer for use in the prosthetic device.

### BACKGROUND

Cartilage may be damaged by direct contact injury, inflammation or, most commonly, by osteoarthritis. Osteoarthritis is a tissue degeneration process that can accompany daily cartilage wear. In osteoarthritis, damage to the articular surface of joints results from the normal aging process or a traumatic injury, typically resulting from high impact loading in work and/or sports, which progressively worsens over time. The injured cartilage goes through several stages of degradation in which the surface softens, flakes and fragments. Finally, the entire cartilage layer is lost and the underlying subchondral bone is exposed. Cartilage does not possess the capacity to heal easily once damaged. There is, therefore, a need to provide prostheses having cartilage regenerative properties.

A number of treatments are available to treat articular cartilage damage in joints, such as the knee, starting with the most conservative, non-invasive options and ending with total joint replacement if the damage has spread throughout the joint. Currently available treatments include anti-inflammatory medications in the early stages. Although anti-inflammatory medications may relieve pain, they have limited effect on arthritis symptoms and further do not repair joint tissue. Cartilage repair methods, such as arthroscopic debridement, attempt to at least delay tissue degeneration. Cartilage repair methods, however, are only partly effective at repairing soft tissue, and do not restore joint spacing or improve joint stability. Joint replacement (arthroplasty) is considered as a final solution, when all other options to relieve pain and restore mobility have failed or are no longer effective. While joint arthroplasty may be effective, the procedure is extremely invasive, technically challenging and may compromise future treatment options. Cartilage regeneration has also been attempted, more, in particular, by tissue-engineering technology. The use of cells, genes and growth factors combined with scaffolds plays a fundamental role in the regeneration of functional and viable articular cartilage. All of these approaches are based on stimulating the body's normal healing or repair processes at a cellular level. Many of these compounds are delivered on a variety of carriers or matrices including, but not limited to, woven polylactic acid based polymers or collagen fibers. Despite various attempts to regenerate cartilage using arthroscopic techniques, such as, for instance, drilling of holes to promote cell infiltration from the bone marrow, a reliable and proven treatment does not currently exist for repairing defects to the articular cartilage.

Because the cartilage layer lacks nerve fibers, patients are often not aware of the severity of the damage. During the final stage, an affected joint consists of bone rubbing against bone, which leads to severe pain and limited mobility. By the time patients seek medical treatment, surgical intervention may be required to alleviate pain and repair the cartilage damage. Prostheses have been developed for the joint in order to avoid or postpone such surgical interventions. These prostheses are often implanted in an early stage of damage and are provided for preventive treatment in order to avoid unnoticed degeneration of the joint.

A known prosthesis is described in U.S. Pat. No. 5,171,322, which discloses a biocompatible, well deformable, flexible, resilient material that is placed in the meniscus and attached to soft tissue surrounding the knee joint. However, the known prosthesis has not been able to achieve the load distribution properties similar to a human meniscus and, moreover, does not help in regenerating possibly damaged cartilage.

A biodegradable polyurethane composition is disclosed in International Patent Publication No. WO 2004/065450. The composition includes a covalently bonded bioactive agent and is biodegradable within a living organism to biocompatible degradation products, including the bioactive agent. The bioactive agent is irreversibly released to affect some biological or chemical activity in the host organism.

A peptide-modified polyurethane composition is disclosed in International Patent Publication No. WO 2005/112974. The composition is prepared by reacting an isocyanate, a chain extender and a peptide. The peptide is, therefore, covalently bonded to the other composition components.

### SUMMARY

The present disclosure describes several exemplary embodiments of the present invention.

One aspect of the present disclosure provides a prosthesis device, comprising a body at least partly formed from a segmented thermoplastic elastomer having crystallized blocks, and having at least one functional component which is able to reversibly bond to the crystallized blocks, wherein the elastomer has cartilage regenerative properties.

Another aspect of the present disclosure provides a method for the preparation of a biocompatible segmented thermoplastic elastomer having crystallized blocks and at least one functional component which is able to reversibly bond to the crystallized blocks, wherein the elastomer has cartilage regenerative properties, the method comprising dissolving the functional component and the elastomer into a solvent; mixing the solution; and at least partly evaporating the solvent.

A further aspect of the present disclosure provides a biocompatible segmented thermoplastic elastomer, comprising crystallized blocks, and at least one functional component which is able to reversibly bond to the crystallized blocks, wherein the elastomer has cartilage regenerative properties and can be used in a prosthesis device able to grow into cartilage.

An additional aspect of the present disclosure provides a method for inserting a prosthesis device into a joint space, comprising providing a prosthesis device comprising a body at least partly formed from a segmented thermoplastic elastomer having crystallized blocks, and having at least one functional component which is able to reversibly bond to the crystallized blocks, wherein the elastomer has cartilage regenerative properties; making an incision in the tissue surrounding the joint space of a knee; inserting the prosthesis device into the joint space of the knee; and closing the incision.

Yet another aspect of the present disclosure provides a method for inserting a prosthesis device into a bone structure, comprising providing a prosthesis device comprising a body at least partly formed from a segmented thermoplastic elastomer having crystallized blocks, and having at least one functional component which is able to reversibly bond to the crystallized blocks, wherein the elastomer has cartilage regenerative properties; making an incision in the tissue surrounding the bone structure; boring a hole into the bone structure; inserting the prosthesis device into the hole; and closing the incision.

It is one feature of the present disclosure to provide a prosthetic device having improved load distribution as well as cartilage regenerating properties.

The prosthetic device according to one exemplary embodiment comprises a body at least partly formed from a biocompatible elastomer, in particular, a segmented thermoplastic elastomer having crystallized blocks, and at least one functional component which is reversibly bonded to the crystallized blocks and has cartilage regenerative properties. The use of a segmented thermoplastic elastomer (hereinafter also referred to as TPE), instead of a chemically crosslinked rubber allows to mould the prosthetic device into the right shape that is individual to the patient. This can, for instance, be carried out by heating since, in TPE, the crosslinks can be broken reversibly as they are of a physical nature. TPE are polymers that combine advantages of both thermoplastic polymers and elastomers. The specific properties of TPE are a result of their morphology. At ambient temperature, the physical crosslinks in the amorphous matrix give the material its elastomeric, rubber-like properties. At higher temperatures, these physical crosslinks are broken (reversibly), and the material can be processed easily, characteristic for thermoplastics. The TPE according to the present disclosure are segmented copolymers, where the reversible physical crosslinks originate from crystallization of one of the blocks of the segmented copolymer. Particularly preferred TPE contain ‘hard’ crystallized blocks of polyester, polyamide and/or polyurethane segments. TPE are used in the prosthetic device of the present disclosure since they combine mechanical stability at low temperatures, i.e., at body temperature, and easy processability and formability at higher temperatures, more, in particular, at temperatures above the melting point of the hard blocks.

One exemplary embodiment of the prosthetic device is characterized in that the segmented thermoplastic elastomer is a thermoplastic elastomeric polyurethane (TPU). The TPU comprises basically three building blocks: a long-chain diol, for example, with a polyether or polyester backbone, a diisocyanate and, finally, a chain extender, such as water, a short-chain diol, or a diamine.

TPU are typically prepared in a one pot procedure, in which the long-chain diol is first reacted with an excess of the diisocyanate, to form an isocyanate functionalized prepolymer. The latter is subsequently reacted with the chain extender which results in the formation of the high molecular weight polyurethane. If a diamine is used as the chain extender, the TPU will also contain urea moieties, which is preferred. At room temperature, the low melting soft blocks are incompatible with the high melting hard blocks, which induces microphase separation by crystallization or liquid-liquid demixing.

The synthetic procedure to prepare TPU generally leads to a distribution in the hard block lengths. As a result, the phase separation of these block copolymers is incomplete. Part of the hard blocks, in particular, the shorter ones, are dissolved in the soft phase, causing an increase in the glass transition temperature, which is undesired for the low temperature flexibility and elasticity of the material. The polydisperse hard block is manifested in a broad melting range and a rubbery plateau in dynamic mechanical thermal analysis (DMTA that is dependent on temperature, i.e., is not completely flat. In order to solve this problem, preferably block copolymers containing hard blocks of substantially uniform length are used in the prosthetic device. Preferred examples of types of hard blocks include, but are not limited to, non-hydrogen bonding polyurethane moieties, polyurethane urea moeities, and aramid moeities. TPE containing substantially uniform hard blocks may be prepared by fractionation of a mixture of hard block oligomers, and subsequent copolymerization of the uniform hard oligomer of a specific length with the prepolymer.

In one exemplary embodiment, the prosthesis comprises a segmented TPE with crystallized blocks comprising bis-urea moieties. TPE with hard blocks based on, preferably uniform, bis-urea moieties have the advantage that their synthesis makes use of simple isocyanate chemistry. These TPE may, for instance, be prepared by a chain extension reaction of an isocyanate functionalized prepolymer with a diamine, or by a chain extension reaction of an amine functionalized prepolymer with a diisocyanate. Examples of suitable, commercially available diamines and diisocyanates include alkylene diamines, diisocyanates, arylene diamines and/or diisocyanates. Amine functionalized prepolymers are also commercially available, or can be prepared from (readily available) hydroxy functionalized prepolymers by cyanoethylation followed by reduction of the cyano-groups, by Gabriel synthesis (halogenation or tosylation followed by modification with phthalimide, and finally formation of the primary amine by deprotection of the phthalimide group) or by other methods that are known in the art. Isocyanate functionalized prepolymers can be prepared by reaction of hydroxy functionalized prepolymers with diisocyanates, such as, for example, isophorone diisocyanate (IPDI), 1,4-diisocyanato butane, 1,6-diisocyanato hexane or 4,4′-methylene bis(phenyl isocyanate). Alternatively, isocyanate functionalized prepolymers can be prepared from amine functionalized prepolymers, for example, by reaction with di-tert-butyl tricarbonate. Hydroxy functionalized prepolymers of molecular weights typically ranging from about 500 g/mol to about 5000 g/mol of all sorts of compositions are also advantageously used. Examples include prepolymers of polyethers, such as polyethylene glycols, polypropylene glycols, poly(ethylene-co-propylene) glycols and poly(tetrahydrofuran), polyesters, such as poly(caprolactone)s or polyadipates, polycarbonates, polyolefins, hydrogenated polyolefins such as poly(ethylene-butylene)s, and the like.

According to one exemplary embodiment, a prosthetic device comprises a body at least partly formed from a biocompatible elastomer, which includes at least one functional component, reversibly bonded to the crystallized blocks, and having cartilage regenerative properties. A particularly preferred functional component comprises a peptide, even more preferred a peptide comprising at least one RGD-sequence, and most preferred a peptide comprising a RGD sequence capable of binding integrins and thereby stimulating cell adhesion; and/or comprising a RGD sequence with specific flanking amino acids such that it contains motifs from extracellular cartilage matrix molecules, such as fibronectin, COMP and/or others. These peptides not only stimulate cell adhesion but preferably also induce proper chondrocyte differentiation such that the synthesis of collagen type 2 may increase and collagen type 1 may decrease. In addition, molecules that induce catabolic effects on the cartilage such as MMPs, ILs and/or TNFs may decrease as well. The peptide sequence is preferably fine tuned such that the newly synthesized cartilage will have optimal mechanical properties that mimic the host cartilage. Peptides comprising at least one RGD-sequence are known per se, but not in the particular combination with the TPE and/or prosthetic device of the present disclosure. In order to incorporate the functional component into the TPE, several possibilities exist. A particularly preferred TPE having at least one functional component comprises uniform bis-urea moieties. TPEs with hard blocks that are based on uniform bis-urea units have an additional advantage due to the presence of these bisurea units and due to the specific morphology of these TPEs. The bis-urea units in the polymer chains stack via (reversible) hydrogen bonding interactions to form the phase separated hard blocks. Due to the uniformity and specific length between the ureas, the bis-urea structural element can be employed as a recognition site for the reversible binding of guest molecules. Functionality can be introduced into the bis-urea stack and, therefore, into the polymer material. This is achieved by adding a functional component, for example, a dye or a peptide, that preferably also bears the specific bis-urea group, for instance, a functionality that bears a bis-ureido-butylene moiety is incorporated into the bis-ureido-butylene stack of a TPE, and is thereby anchored into the polymer material.

According to the present disclosure, related to a prosthetic device for the human body, it is particularly preferred that peptides with a certain specific function (promotion of cell binding, promotion of cell growth, etc.) are modularly added to the TPE of choice. Thereby a biofunctional, and biocompatible material may be obtained. Particularly preferred is a prosthesis, wherein the at least one functional component comprises a peptide. Even more preferred is a prosthesis, wherein the peptide comprises at least one RGD-sequence. Most preferred is a peptide comprising a RGD sequence capable of binding integrins and thereby stimulating cell adhesion; and/or comprising a RGD sequence with specific flanking amino acids such that it contains motifs from extracellular cartilage matrix molecules, such as fibronectin, COMP and/or others. These peptides not only stimulate cell adhesion but preferably also induce proper chondrocyte differentiation such that the synthesis of collagen type 2 may increase and collagen type 1 may decrease. In addition, molecules that induce catabolic effects on the cartilage such as MMPs, ILs and/or TNFs may decrease as well. The peptide sequence is preferably fine tuned such that the newly synthesized cartilage will have optimal mechanical properties that mimic the host cartilage. This readily promotes growth of cartilage cells of hyaline type, which results in strong and wear resistant cartilage.

An aspect of the prosthetic device is its ability to grow into cartilage and effect cartilage regeneration. Tissue engineering methods in which, prior to introduction of a prosthesis in a host organism, cells are cultivated on the surface of the prosthesis in order to improve biocompatibility, are not needed.

The prosthetic device can deform to distribute the physiologic loads over a large area such that the joint space is maintained under physiologic loads. The body of the prosthesis preferably has a shape that is contoured to fit with the femoral condyle, the tubercle, and the tibial plateau but is allowed to translate within the joint space. As is well known by those skilled in the art, the femoral condyle, tubercle, and tibial plateau of a given knee may vary in shape and size. As such, while various specific shapes are shown and described herein, it should be understood that various other shapes and configurations are within the scope of the present disclosure. Moreover, the prosthetic device is preferably used without any means of attachment and remains in the joint space by its geometry and the surrounding soft tissue structures. The prosthesis can also be used for other joint spaces, such as a temporal-mandibular joint, an ankle, a hip, a shoulder, for instance. The use of the segmented elastomer in the prosthesis of the present disclosure yields a compliant, wear-resistant prosthesis, having load distribution capabilities similar to native articular cartilage and meniscus.

In another exemplary embodiment of the prosthesis, the body further comprises a reinforcing material selected from the group consisting of polymers and/or metals. In an even more preferred exemplary embodiment, the reinforcing material is a foam, preferably a metal foam. In a particularly preferred exemplary embodiment the foam forms the core of the body and the elastomer skin. This exemplary embodiment allows bone in-growth into the foam, whereby a strong fixation is build between prosthesis and bone. Cartilage cells from the host cartilage having a strong affinity for the segmented elastomer skin of the body, will colonise the surface thereof and will be triggered by the polymer with its peptides to produce new hyaline cartilage tissue.

In another exemplary embodiment of the prosthesis, the RGD sequence containing peptides, optionally having flanking amino acids, should stand out over some distance from the surface of the segmented thermoplastic elastomer to further the cell adhesive properties. This can be achieved, for instance, by adding Glycines to the peptide. The spacer preferably has a length in the order of 2-30 glycine molecules (corresponding to about 7-100 angstrom). The surface density of the active molecule that is binded to the elastomer can become active for values as low as 10 fmol/cm² but is preferably in the order of 1-10 pmol/cm² to have optimal binding and regulatory effects. To prevent the functional component from negatively affecting the (mechanical) properties of the elastomer, the preferred amount of the functional component, which preferably is a peptide, with respect to the total amount of bis-urea moieties in the elastomer, is lower than 50 mol %, more preferably lower than 30 mol %, and most preferably lower than 20 mol %.

The present disclosure also relates to a method for placing a prosthesis into a joint space. The method comprises making an incision in the tissue surrounding the joint space of a knee; inserting a prosthesis according to the present disclosure into the joint space of the knee; and closing the incision. Another exemplary embodiment of a method according to the present disclosure comprises making an incision in the tissue surrounding the joint space and drilling a hole through the damaged cartilage into the subchondral bone and inserting the prosthesis according to the present disclosure into the joint space, and closing the incision.

The present disclosure also relates to a method for placing a prosthesis into a bone structure. One exemplary embodiment of the method comprises making an incision in the tissue surrounding the bone structure; boring a hole into the bone structure; inserting a prosthesis according to the present disclosure into the hole; and closing the incision. The latter method is particularly useful in combination with a prosthesis of which the body comprises a foam core and a segmented copolymer TPE skin.

### BRIEF DESCRIPTION OF THE DRAWINGS

Various aspects of the present disclosure are described hereinbelow with reference to the accompanying figures, in which like reference characters refer to like parts throughout the several views, of which:

FIG. 1 is a schematic view from above of an exemplary medial meniscus prosthesis;

FIG. 2 is a schematic side view along the line AA′ of the embodiment shown in FIG. 1;

FIG. 3 is a schematic side view along the line BB′ of the embodiment shown in FIG. 1;

FIG. 4 is a schematic representation of a TPU and its building blocks; in this case the chain extender is a short diol; and

FIG. 5 is a schematic representation of the morphology of the segmented copolymer TPE.

### DETAILED DESCRIPTION

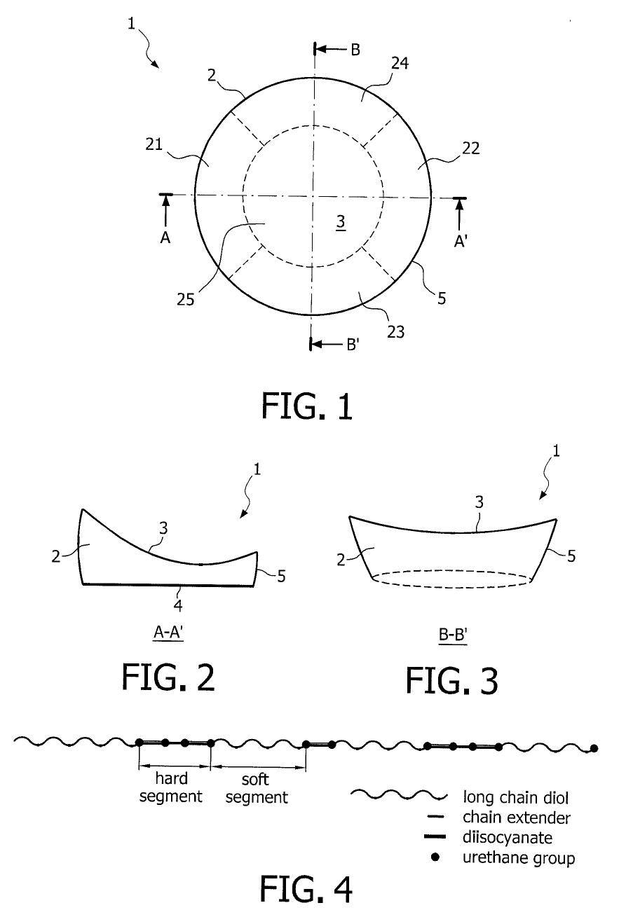

Referring to FIGS. 1, 2 and 3, a prosthesis 1, generally elliptical in shape, comprising a body 2 formed from a segmented copolymer TPE is shown. In one exemplary embodiment, the prosthesis 1 is kidney shaped, but other shapes may be used as well. In particular, the body 2 may be toroidal, circular, planar, donut shaped or crescent shaped. The prosthesis 1 is intended for use in a medial compartment of a knee. It should be understood that a device according to the present disclosure may have a shape being the mirror image of the device illustrated in FIG. 1, depending on which knee is contemplated. The body 2 of the prosthesis 1 has a superior surface 3, an inferior surface 4, and an outer wall 5. The superior surface 3 generally forms a concave surface, contoured to fit with a femoral condyle while the inferior surface 4 forms a generally convex surface, contoured to fit on top of a tibial plateau. As shown in FIG. 3, the inferior surface 4 may also be shaped concavely. The body 2 further includes a cruciate region 21, an outer region 22, an anterior region 23, a posterior region 24 and a central region 25. The outer wall 5 is formed from the periphery of the cruciate region 21, outer region 22, anterior region 23 and posterior region 24. The various regions are contiguous but may not be clearly delineated. Instead, the regions are defined merely to provide a point of reference for various aspects of the present disclosure. The prosthesis 1 is wide enough to fully receive the width of the femoral condyle. The length of the prosthesis 1 is approximately equal to the anterior-posterior length of the tibial plateau. By being wider, the prosthesis 1 is able to provide a channel to guide the femoral condyle, aiding the prosthesis 1 to maintain its position within the space between two bones (“joint space”) during kinematic joint motion of the knee. By simultaneously having a preferably convex inferior surface 4, contoured to receive the tibial plateau, the prosthesis 1 maintains its position within the joint space. While a secure fit within the joint space is needed, it should be understood that the prosthesis 1 may shift slightly or translate during movement of the joint. In relation to the knee joint, the prosthesis 1 must, for instance, be able to engage in natural motion, including flexion and extension motions commonly associated with typical movement, without unrecoverably unseating from the tibial plateau. As used herein, “unrecoverably unseating” refers to a shift in the positioning of the device that is so significant that it is unable to return to its original position. As is clear from FIG. 3, the posterior region 24 has a slightly greater thickness than the anterior region 23. The greater thickness of the prosthesis 1 at its posterior region 24 aids the prosthesis 1 to stay in place by forming a barrier to anterior displacement through the joint space. The greater thickness of the posterior region 24, however, does not pose a problem during insertion due to the compliant nature of the segmented elastomer. Generally the thickness of the posterior region 24 ranges between about 2 and 15 mm while the anterior region 23 ranges between 1 and 12 mm. The cruciate region 21, the outer region 22, and the central region 25 may have thicknesses ranging from 1 to 20 mm. A prosthesis 1 according to the present disclosure may include one or more sloped areas in the various regions and surfaces to enable the prosthesis 1 to stay on the tibial plateau during flexion and extension without the need for any additional securing means. Specifically, the geometry of the prosthesis 1 is selected to enable the body 2 to fit between the tibial plateau and the femoral condyle while taking into account the tubercle without the need for cement, pinning or other surgical securement means. While preferably the prosthesis 1 does not require a means of attachment beyond its geometry, a tissue fixation component, such as tabs or holes to allow the surgeon to suture the prosthesis 1 to native body structures, may be combined with the prosthesis 1 to enhance tissue fixation.

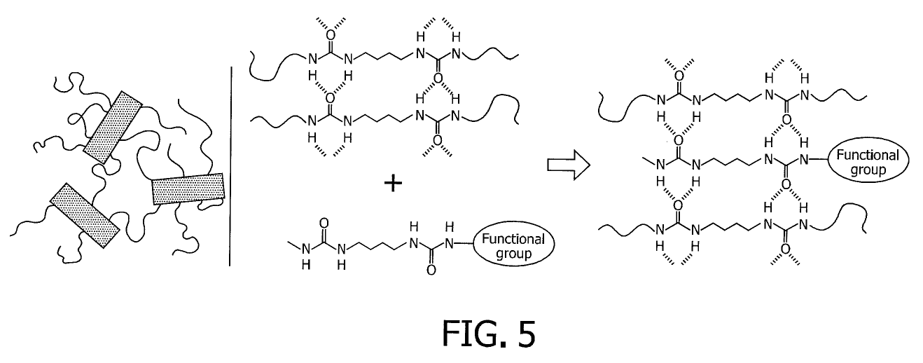

According to the present disclosure the prosthesis is made of segmented thermoplastic elastomer having crystallized blocks and at least one functional component which is able to reversibly bond to the crystallized blocks and has cartilage regenerative properties. An example of a preferred TPU and its building blocks is shown in FIG. 4. In the exemplary embodiment shown, the chain extender is a short diol. In FIG. 5, a schematic representation of the morphology of the segmented copolymer TPE according to the present disclosure is shown. The depicted TPE with hard blocks based on uniform bis-urea units have the desired properties due to the presence of the bisurea units and due to the specific morphology of these TPE. Referring to FIG. 5 (left side) the bis-urea units in the polymer chains stack via (reversible) hydrogen bonding interactions to form the phase separated hard blocks. Due to the uniformity and specific length between the ureas, the bis-urea structural element can be employed as a recognition site for the reversible binding of guest molecules. Functionality can be introduced into the bis-urea stack and, therefore, into the segmented elastomer. This is achieved by adding a peptide that also bears the specific bis-urea group. This is shown in FIG. 5 on the right: a functionality that bears a bis-ureido-butylene moiety is incorporated into the bis-ureido-butylene stack of the TPE and is thereby anchored into the elastomer. According to the present disclosure, peptides with a certain specific function (promotion of cell binding, promotion of cell growth, etc.) can be modularly added to the TPE, thereby making a biofunctional material. A particularly preferred prosthesis comprises a peptide comprising at least one RGD-sequence. Most preferred is a peptide comprising a RGD sequence capable of binding integrins and thereby stimulating cell adhesion; and/or comprising a RGD sequence with specific flanking amino acids such that it contains motifs from extracellular cartilage matrix molecules, such as fibronectin, COMP and/or others.

According to one aspect of the present disclosure, a method is provided for the preparation of a biocompatible elastomer, in particular, a segmented thermoplastic elastomer having crystallized blocks, and at least one functional component, which is able to reversibly bond to the crystallized blocks, and has cartilage regenerative properties, the method comprising dissolving the functional component and the elastomer into a solvent, mixing the solutions and at least partly evaporating the solvent. In the thus obtained biocompatible elastomer, the functional component, which is preferably selected as described above, is reversibly bonded to the elastomer, preferably by reversible bonding to the phase separated hard blocks of the elastomer.

Aspects of the disclosure will be further described in connection with the following examples, which are set forth for purposes of illustration only. Parts and percentages appearing in such examples are by weight unless otherwise stipulated.

### EXAMPLES

### Materials Used

Bis(3-aminopropyl)-poly(tetrahydrofuran) with molecular weight 1100 g/mol and hydroxy terminated poly(tetrahydrofuran) with molecular weight 2000 g/mol were purchased from Aldrich. Hydroxy terminated random copolymer of THF (tetrahydrofuran) and EO (ethylene oxide) of molecular weight 4000 g/mol was kindly provided by Akzo-Nobel (ca. 10% of the monomeric units are EO), and hydroxy-terminated poly(ethylene-ran-butylene) (hydrogenated polybutadiene, Kraton liquid polymer L-2203, Mn=3500 g/mol) was kindly provided by Kraton Polymers Research. 1,4-Diisocyanatobutane, 1,3-phenylenediisocyanate, 4,4′-methylenebis(phenylene diisocyanate), borane-tetrahydrofuran complex (1 M in THF), and sodium hydride (60% dispersion in mineral oil) were purchased from Aldrich. 1,2-Ethylenediamine was purchased from Acros. 1,6-Diisocyanatohexane was purchased from Fluka. di-tert-Butyl tricarbonate was prepared according to literature proceedings (Peerlings, H. W. I. and Meijer, E. W., Tetrahedron Letters, 1999, 40, 1021), as well as N-carbobenzoxy-6-aminohexanoic acid (Shah, J. et al., J. Med. Chem., 1995, 38, 4284). Poly(ε-caprolactone)diol (Mn=1250 and 2000 g/mol), dicyclohexylcarbodiimide (DCC), p-toluenesulphonic acid·H₂O and 4-(N,N′-dimethyl)aminopyridine (DMAP) were purchased from Acros. Sodium hydroxide (NaOH), 4 Å molsieves and Pd/C(10%) were purchased from Merck. Dibutyltin dilaurate, 1,4-diaminobutane and hexylamine were purchased from Aldrich. Sodium dodecyl sulfate (SDS), 1-hydroxybenzotriazole hydrate (HOBt), diisopropylcarbodiimine (DIPCDI) and 6-(Fmoc-amino)caproic acid were purchased from Fluka. Wang-resin (D-1250) loaded with 0.63 mmol gram⁻¹ FMOC protected serine (FMOC-Ser(tBu)), FMOC-Asp(OtBu), FMOC-Glycine and FMOC-Arg(PMC) were purchased from Bachem. All solvents were purchased from Biosolve. Deuterated solvents were purchased from Cambridge Isotope Laboratories. Water was always demineralized prior to use. Chloroform was dried over molsieves. Further chemicals were used without further purification. All reactions were carried out under a dry argon atmosphere, except for the synthesis of the peptide.

### Equipment Used

Infra red spectra were measured on a Perkin Elmer Spectrum One FT-IR spectrometer with a Universal ATR Sampling Accessory. ¹H-NMR and ¹³C-NMR spectra were recorded on a Varian Gemini 300 MHz or a Varian Mercury 400 MHz NMR spectrometer. Molecular weights of the synthesized polycaprolactone polymers were determined by size exclusion chromatography (SEC) using a poly(styrene) calibrated PL-SEC 120 high temperature chromatograph that was equipped with a PL gel 5 μm mixed-C column, an autosampler and an RI detector at 80° C. in 1-methyl-2-pyrrolidinone (NMP). The poly(tetrahydrofuran) polymers were analyzed with SEC on a Shimadzu LC 10-AT, using a Polymer Laboratories Plgel 5 μm mixed-D column, a Shimadzu SPD-10AV UV-Vis or a Shimadzu RID-6S detector, and NMP as eluent; polystyrene standards were used for calibration. Differential Scanning Calorimetry (DSC) measurements were performed on a Perkin Elmer Differential Scanning Calorimeter Pyris 1 with Pyris 1 DSC autosampler and Perkin Elmer CCA7 cooling element under a nitrogen atmosphere. Melting and crystallization temperatures were determined in the second heating run at a heating/cooling rate of 10° C. min⁻¹, glass transition temperatures at a heating rate of 40° C. min⁻¹. Optical properties and flow temperatures were determined using a Jeneval polarization microscope equipped with a Linkam THMS 600 heating device with crossed polarizers. MALDI-TOF spectra were obtained on a Perseptive Biosystems Voyager DE-Pro MALDI-TOF mass spectrometer (accelerating voltage: 20kV; grid voltage: 74.0%, guide wire voltage: 0.030%, delay: 200 ms, low mass gate 900 amu). Samples for MALDI-TOF were prepared by adding a solution of the polymers in THF (20 μl, c=1 mg/ml) to a solution of α-cyano-4-hydroxycinnamic acid in THF (10 μl, c=20 mg/ml) and subsequent thoroughly mixing. This mixture (0.3 μl) was brought on a sample plate, and the solvent was evaporated. Reversed phase liquid chromatography—mass spectroscopy (RPLC-MS) was performed on a system consisting of the following components: Shimadzu SCL-10A VP system controller with Shimadzu LC-10AD VP liquid chromatography pumps with an Alltima C 18 3u (50 mm×2.1 mm) reversed phase column and gradients of water-acetonitrile-isopropanol (1:1:1 v/v supplemented with 0.1% formic acid), a Shimadzu DGU-14A degasser, a Thermo Finnigan surveyor autosampler, a Thermo Finnigan surveyor PDA detector and a Finnigan LCQ Deca XP Max.

### Example 1

### [pTHF₁₁₀₀-U-C₄H₈-U]ₙ, With U representing a Urea Group

Bis(3-aminopropyl)-poly(tetrahydrofuran), Mₙ=1100 g/mol, (10.00 g, 9.09 mmol) was dissolved in chloroform (100 ml), and to this solution a solution of 1,4-diisocyanatobutane (1.4 g, 9.99 mmol) in chloroform (40 ml) was added dropwise. The mixture was stirred for 1 h, and subsequently partly concentrated, and methanol (5 ml) was added. The product was precipitated in hexane (500 ml), filtered and dried in vacuo. It was obtained as white, fluffy, elastic fibers (10.62 g, 93%). ¹H-NMR (CDCl₃): δ 5.4-4.8 (4H, NH), 3.41 (58H, CH₂O), 3.25 (4H, OCH₂CH₂CH₂N), 3.17 (4H, NCH₂CH₂CH₂CH₂N), 1.74 (4H, OCH₂CH₂CH₂N), 1.62 (58H, OCH₂CH₂CH₂CH₂O), 1.50 (4H, NCH₂CH₂CH₂CH₂N). FT-IR (ATR): ν 3324 (N-H stretching), 2940, 2854, 1615 (C=O stretching), 1580 1365, 1104 (C—O stretching) cm⁻¹. SEC (NMP, rel. to PS): Mₙ=42*10³ g/mol. DSC: Tg=−68° C., Tm=102° C. T-flow=140° C.

### Example 2

### [pTHF₁₁₀₀-U-X-U]ₙ, With X=n-C₆H₁₂, Metha-Ph or Para-(Ph—CH₂—Ph)

In a similar way as in Example 1, bis(3-aminopropyl)-poly(tetrahydrofuran) Mₙ=1100 g/mol was reacted at room temperature with 1 molar equivalent of 1,6-diisocyanatohexane (X=n-C₆H₁₂), 1,3-phenylenediisocyanate (X=metha-Ph) or 4,4′-methylenebis(phenyl isocyanate) (X=para-(Ph—CH₂—Ph)), using chloroform as reaction solvent. After isolation by precipitation and drying, the polymer products had molecular weights of Mₙ=43* 103 g/mol (X=n-C₆H₁₂), 38*103 g/mol (X=metha-Ph) and 55*103 g/mol (para-(Ph—CH₂—Ph)) as measured with SEC using NMP as eluent and relative to polystyrene standards. The isolated polymers were all three obtained as highly elastic fluffy materials, and could be solvent casted from chloroform to obtain a transparent elastic film after evaporation of the solvent.

### Example 3

### [pTHF₁₁₀₀-U-C₂H₄-U]ₙ

Bis(3-aminopropyl)-poly(tetrahydrofuran), Mₙ=1100 g/mol, (0.50 g, 0.45 mmol) was dissolved in chloroform (10 ml), and a solution of di-tert-butyl tricarbonate (0.235 g, 0.91 mmol) in chloroform (1 ml) was injected into this solution. The reaction mixture was stirred for 30 min. during which time the amines were converted to isocyanate groups. Then, 1,2-ethylenediamine (0.0269 g, 0.45 mmol) in chloroform (3 ml) was added dropwise, and the solution was stirred for 1 h, and subsequently partly concentrated, and methanol (1 ml) was added. The product was precipitated in pentane (50 ml), filtered and dried in vacuo. The product was obtained as white, fluffy, elastic fibers (0.47 g, 86%). ¹H-NMR (DMSO): δ 5.91 (4H, NH), 3.34 (59H, CH₂O), 2.99 (8H, CH₂N), 1.51 (56H, CH₂CH₂CH₂). FT-IR (ATR): ν 3329 (N—H stretching), 2937, 2854, 1615 (C=O stretching), 1589 1366, 1105 (C—O stretching) cm⁻¹. SEC (NMP, rel. to PS): Mₙ=41*10³ g/mol. T-flow=115° C.

### Example 4

### Bis(2-cyanoethyl)-poly(tetrahydrofuran), Mₙ=2000 g/mol

Poly(tetrahydrofuran) diol, Mₙ=2000 g/mol, (20.00 g; 10.0 mmol) and 15-crown-5 (44 mg; 0.2 mmol) were dissolved in acrylonitrile (40 ml) and cooled on an icebath. Sodium hydride (8 mg 60% dispersion in mineral oil; 0.2 mmol) is added to the solution, and the reaction mixture is stirred at 0° C. for about 15 min, after which the reaction mixture turned slightly yellow. At this point, the reaction was quenched by addition of a drop of concentrated hydrochloric acid. The solution was concentrated, the residue taken up in dichloromethane (100 ml) and centrifuged at 4500 rpm. The mixture was decanted, filtered, and concentrated in vacuo. The product was obtained as a slightly yellow, viscous liquid, that slowly crystallized (20.13 g, 96%). ¹H-NMR (CDCl₃): δ 3.62 (t, 4H, OCH₂CH₂CN), 3.51 (t, 4H, CH₂OCH₂CH₂CN), 3.40 (br. t, 106H, OCH₂CH₂CH₂CH₂O main chain), 2.59 (t, 4H, CH₂CN), 1.60 (br. m, 110H, OCH₂CH₂CH₂CH₂O main chain). ¹³C-NMR (CDCl₃): δ 117.7 (CN), 71.0 (CH₂OCH₂CH₂CN), 70.4 (OCH₂CH₂CH₂CH₂O main chain), 65.1 (OCH₂CH₂CN), 26.3 (OCH₂CH₂CH₂CH₂O main chain), 18.7 (CH₂CN). FT-IR (ATR): ν 2939, 2855, 2161 (w, C≡N stretching), 1367, 1103 (C—O stretching) cm⁻¹.

### Example 5

### Bis(3-aminopropyl)-poly(tetrahydrofuran), Mₙ=2000 g/mol

To a solution of borane-tetrahydrofuran complex (80 ml IM in THF, 80 mmol) in dry THF (240 ml) was added slowly bis(2-cyanoethyl)-poly(tetrahydrofuran) of Example 4 (20.00 g, 9.5 mmol) dissolved in dry THF (160 ml) at 0° C. The solution was stirred for 30 min at 0° C., after which it was heated to reflux for 4 h. The reaction mixture was cooled to 0° C., and methanol (80 ml) was added dropwise. (Be careful: hydrogen-gas evolution). Hydrochloric acid (4 ml, 37% in water) was added slowly, and the reaction mixture was stirred for 1 h, and subsequently evaporated to dryness under reduced pressure. Trimethyl borate was removed by three coevaporations with methanol (3 times 100 ml). To the viscous liquid was added sodium hydroxide solution (150 ml, 1M in water), and this was extracted with diethyl ether (3 times 300 ml). The combined organic layers were dried with sodium sulfate, filtered, and the solvent was evaporated on a rotary evaporator without putting the flask in the water bath. During the evaporation, the polymer precipitated from the cold solution and was obtained as a white powder (18.74 g, 93%). ¹H-NMR (CDCl₃): δ 3.49 (t, 4H, OCH₂CH₂CH₂NH₂), 3.41 (br. t, 138H, OCH₂CH₂CH₂CH₂O main chain), 2.79 (t, 4H, CH₂NH₂), 1.71 (t, 4H, OCH₂CH₂CH₂NH₂), 1.62 (br. m, 142H, OCH₂CH₂CH₂CH₂O main chain), 1.1 (br. s, 4H, NH₂). ¹³C-NMR (CDCl₃): δ 70.5 (OCH₂CH₂CH₂CH₂O main chain), 68.8 (OCH₂CH₂CH₂NH₂), 39.7 (CH₂NH₂), 33.6 (OCH₂CH₂CH₂NH₂), 26.4 (OCH₂CH₂CH₂CH₂O main chain). FT-IR (ATR): ν 3564, 3539, 2941, 2862, 1492, 1372, 1107, 996 cm⁻¹. MALDI-TOF [M+Na⁺]=Calcd. 155.1+n*72.0 Da. Obsd. 155.9+n*72.0 Da. SEC (phenyl urea derivative): Mₙ=3769 g/mol, PDI=1.5. Mₙ according to ¹H NMR: 2500 g/mol.

### Example 6

### [pTHF₂₀₀₀-U-C₄H₈-U]ₙ

In a similar way as described in Example 1 for bis(3-aminopropyl)-poly(tetrahydrofuran) Mₙ=1100 g/mol, bis(3-aminopropyl)-poly(tetrahydrofuran) Mₙ=2000 g/mol from Example 5 was reacted at room temperature with 1 molar equivalent of 1,4-diisocyanatobutane using chloroform as reaction solvent. After similar work-up as described in Example 1, the isolated polymer product was obtained as a white elastic fluffy material. The polymer product had a molecular weight of Mₙ=53*10³ g/mol as measured with SEC using NMP as eluent and relative to polystyrene standards. DSC: Tg=−74° C., Tm1=1° C., Tm2=101° C. T-flow=125° C.

### Example 7

### Bis(3-aminopropyl)-poly(tetrahydrofuran-ran-ethyleneoxide), Mₙ=4000 g/mol

In a similar way as described in examples 4 and 5 for hydroxy terminated poly(terahydrofuran) Mₙ=2000 g/mol, hydroxy terminated poly(tetrahydrofuran-ran-ethylene oxide) Mₙ=4000 g/mol was transformed to its bis(3-aminopropyl) analogue. Briefly, first the hydroxy terminated polymer was cyanoethylated, and then the resulting cyano terminal groups were reduced to primary amine groups using borane. Mₙ according to ¹H NMR: 4500 g/mol.

### Example 8

### [p(THF-EO)₄₀₀₀-U-C₄H₈-U]ₙ

In a similar way as described in Example 1 for bis(3-aminopropyl)-poly(tetrahydrofuran) Mₙ=1100 g/mol, bis(3-aminopropyl)-poly(tetrahydrofuran-ran-ethylene oxide) Mₙ=4000 g/mol from Example 7 was reacted at room temperature with 1 molar equivalent of 1,4-diisocyanatobutane using chloroform as reaction solvent. After similar work-up as described in Example 1, the isolated polymer product was obtained as a white elastic fluffy material. The polymer product had a molecular weight of Mₙ=58*10³ g/mol as measured with SEC using NMP as eluent and relative to polystyrene standards. DSC: Tg=−73° C., Tm1=1° C., Tm2=48° C. T-flow=140° C.

### Example 9

### [pCL₂₀₀₀-Urethane-Urea]ₙ

Poly(ε-caprolactone) diol (10 g, 5 mmol, Mₙ=2000 g/mol) was dissolved in 100 mL of CHCl₃, dried over MgSO₄ and filtered during transfer to the reaction flask. Under an argon atmosphere, 1,4-diisocyanatobutane (1.9 mL, 15 mmol) and 4 drops of dibutyltin dilaurate were added to this solution. This solution was refluxed overnight at 85° C. under argon. After precipitation in heptane a white powder in a yield of 80% was obtained. This isocyanate-functionalized polycaprolactone (9.7 g, 4.2 mmol) was then dissolved in 200 mL dry CHCl₃. Subsequently 1,4-diaminobutane (0.42 mL, 4.2 mmol) was dissolved in 50 mL dry CHCl₃ and slowly added drop wise to the first solution until the isocyanate signal in FT-IR had disappeared. Precipitation in hexane resulted in a white flaky solid in 75% overall yield.

FT-IR: ν=3326, 2943, 2866, 1723, 1680, 1623, 1575, 1538 cm⁻¹. ¹H-NMR (CDCl₃/MeOD): δ=5.2-5.0 (b, 6H), 4.23 (t, 4H), 4.06 (t, 2(2n)H), 3.70 (t, 4H), 3.16 (b, 12H), 2.31 (t, 2(2n)H), 1.65 (m, 2(4n)H), 1.51 (m, 12H), 1.40 (m, 2(2n)H) ppm, with n≈17. ¹³C-NMR (CDCl₃): δ=173.5, 68.7, 63.9, 63.0, 40.0, 39.7, 33.7, 28.3, 26.9, 26.7, 27.9, 25.1, 24.2 ppm. SEC: Mₙ=86 kg/mole, Mₙ=192 kg/mole, PDI=2.2. DSC: Tg=−50° C., Tm=42° C.

### Example 10

### [pCL₁₂₅₀-Urethane-Urea]ₙ

This polymer was synthesized in a manner similar to that used for [pCL₂₀₀₀-Urethane-Urea]ₙ, except that polycaprolactone of Mₙ=1250 g/mol was used as starting material. Overall yield=56%, FT-IR, ¹H-NMR (CDCl₃/MeOD) and ¹³C-NMR (CDCl₃) similar to that of [pCL₂₀₀₀-Urethane-Urea]ₙ. DSC: Tg=−53° C., Tm=9° C.

### Example 11

### [pCL₂₀₀₀-U-C₄H₈-U]ₙ

Poly(ε-caprolactone) diol (Mₙ=2000, 10 g, 5 mmol), N-carbobenzoxy-6-aminohexanoic acid (2.8 g, 11 mmol), 4-(dimethylamino)pyridinium 4-toluenesulfonate (DPTS) (0.7 g, 2.5 mmol) and DCC (3 g, 15 mmol) were dissolved in CHCl₃ and the reaction was allowed to stir for 48 hours. The reaction mixture was filtered and the solvent was evaporated. The remaining solid material was dissolved in 100 mL CHCl₃ and precipitated in hexane. To remove the remaining DPTS, the solid product was stirred in MeOH. After removing the MeOH, pCL₂₀₀₀ modified with N-carbobenzoxy groups was obtained as a white powder in a 64% yield. A solution of this polymer (4 g, 1.6 mmol) in 100 mL EtOAc/MeOH (v/v 2:1) and 400 mg of 10% Pd supported on activated carbon was subjected to hydrogenation under a H₂ blanket at room temperature for 4 hours. After filtration over Celite, the product was isolated after precipitation in hexane as a white powder in a 95% yield. This intermediate product, pCL₂₀₀₀ modified with primary amine groups (14 g, 6.35 mmol), was dissolved in 100 mL CHCl₃. A solution of 560 μL 1,4-diisocyanatobutane in 5 mL CHCl₃ was slowly added by drops until the signal corresponding to amino methylene protons were no longer visible in ¹H-NMR. The product was isolated in a 58% overall yield by precipitation in hexane. FT-IR: ν=3332, 2942, 2866, 1723, 1620, 1575 cm⁻¹. ¹H-NMR (CDCl₃): δ=5.0-4.8 (b, 4H), 4.23 (t, 4H), 4.07 (t, 2(2n)H), 3.70 (t, 4H), 3.18 (b, 8H), 2.31 (t, 2(2n+2)H), 1.68 (m, 2(4n+2)H), 1.53 (m, 8H), 1.37 (m, 2(2n+2)H) ppm, with n≈17. ¹³C-NMR (CDCl₃): δ=173.5, 158.8, 69.0), 64.1, 63.2, 40.1, 39.8, 34.1, 29.9, 28.3, 27.5, 26.3, 25.5, 24.5 ppm. SEC: Mₙ=34 kg/mole, M_{w}=102 kg/mole, PD=3.0. DSC: Tg=−54° C., Tm1=27° C., Tm2=98° C.

### Example 12

### [pCL₁₂₅₀-U-C₄H₈-U]ₙ

This polymer was synthesized in a manner similar to that used for [pCL₂₀₀₀-U-C₄H₈-U]ₙ. Overall yield=31%, FT-IR, ¹H-NMR (CDCl₃/MeOD) and ¹³C-NMR (CDCl₃) similar to that of [pCL₂₀₀₀-U-C₄H₈-U]ₙ. SEC: Mₙ=56 kg/mole, M_{w}=109 kg/mole, PD=1.9. DSC: Tg=−55° C., Tm1=19° C., Tm2=103° C.

### Example 13

### [pEthylene-Butylene₃₅₀₀-Urethane-Urea]ₙ

Hydroxy-terminated poly(ethylene-ran-butylene) (hydrogenated polybutadiene, Kraton liquid polymer L-2203) (11.55 g) in 20 ml of CHCl₃ was added drop wise over a period of two hours to a solution of isophorone diisocyanate (IPDI, 1.5 g) and a few drops of dibutyl tin laurate in 5 mL of CHCl₃. The solution was stirred overnight under an argon atmosphere. Then, the solution was heated to 60° C. and was stirred for 2 hours. The mixture was cooled again to room temperature and 1,4-butyldiamine (0.3 g) in 3 ml of CHCl₃ was added drop wise. The mixture was stirred overnight, after which completion of the reaction was confirmed by FT-IR analysis (no or hardly any isocyanate resonance peak was present). The material was isolated by precipitation into methanol, and subsequent drying. The material is highly elastic.

### Example 14

### Mechanical Properties as Measured by Tensile Testing

Stress-strain measurements (tensile tests) were performed on a Zwick Z010 Universal Tensile Tester equipped with a 2.5 kN load cell at an elongation rate of 100% per minute. Tensile bars were punched from a solution-cast film of the polymers. The films of the polycaprolactone polymers from Examples 9-12 were thermally annealed at 80° C. or 100° C. Typical dimensions of the tensile bars: length=22 mm, width=5.0 mm, and thickness=0.30 mm. Due to the shape of the curves, yield stresses were determined by determining the intersection point of the two tangents to the initial and final parts of the load elongation curves. An indicative Young's modulus was determined by calculating the slope at zero strain. The following Table shows the tensile testing data as recorded for the given polymer materials.

| | | Young's | Yield | | Strain | Tough- |
|---|---|---|---|---|---|---|
| | | modulus | stress | Strength | at break | ness |
| Example | Polymer | E (MPa) | σ_{y}(MPa) | σ_{br}(MPa) | λ_{br}(%) | (kJ/kg) |
| 1 | pTHF₁₁₀₀-U₂ | 96 | 9.6 | 28 | 1060 | 178 |
| 6 | pTHF₂₀₀₀-U₂ | 26 | 4.7 | 28 | 1175 | 147 |
| 8 | p(THF-EO)₄₀₀₀-U₂ | 11 | 2.4 | 11 | 2140 | 122 |
| 9 | pCL₂₀₀₀-Ur-U₂ | 16 | 2.6 | 30 | 1114 | n.d. |
| 10 | pCL₁₂₅₀-Ur-U₂ | 39 | 8.7 | 18 | 700 | n.d. |
| 11 | pCL₂₀₀₀-U₂ | 14 | 2.6 | 16 | 1330 | n.d. |
| 12 | pCL₁₂₅₀-U₂ | 11 | 3.0 | 21 | 1505 | n.d. |

In DMTA-analysis (1 Hz, 1° C./min heating rate), the polymers of Examples 1, 6 and 8 show rubber plateaus at E′=135 MPa, 17 MPa and 11 MPa, respectively. Flow is achieved at 148° C., 112° C. and 105° C., respectively.

### Example 15

### The Synthesis of Aza-Dyes 15A and 15B

4-Isocyanato-4′-nitroazobenzene.

Disperse Orange 3 (4-(4-Nitro-phenylazo)-aniline) (0.50 g, 2.07 mmol) was dissolved in THF (40 ml), and phosgene (2.2 ml 20% in toluene, 4.1 mmol) was added. The reaction mixture was heated to reflux temperature and stirred for 1 h, while argon was bubbled through the solution. It was evaporated to dryness, and the product was obtained as a red solid (0.62 g, 112%). FT-IR (ATR): ν 2257 (NCO), 1734 (NHCOCl). cm⁻¹.

Aza-dye 15A: 3-(2-ethyl-hexyl)-1-[4-(4-nitro-phenylazo)-phenyl]-urea

4-Isocyanato-4′-nitroazobenzene (0.31 g, 1.00 mmol) was dissolved in THF (15 ml), and 2-ethylhexylamine (0.20 g, 1.5 mmol) in THF (5 ml) was added. The reaction mixture was stirred at room temperature for 30 min, after which it was evaporated to dryness. The product was redissolved in chloroform (20 ml), and extracted with hydrochloric acid solution (10 ml 0.1 M in water), and saturated sodium bicarbonate solution (10 ml). The organic layer was dried with sodium sulfate, filtered and purified by column chromatography using 1% methanol in chloroform as the eluent (R_{f}=0.4). The product was obtained as an orange solid (0.30 g, 75%). ¹H-NMR (DMSO-d6): δ 9.02 (s, 1H, Ph-NH), 8.41 (d, 2H, C2′H, J=9.2 Hz), 8.01 (d, 2H, C3′H, J=8.8 Hz), 7.91 (d, 2H, C2H, J=8.8 Hz), 7.65 (d, 2H, C3H, J=8.8 Hz), 6.35 (t, 1H, CH₂NH), 3.08 (q, 2H, CH₂NH, J=5.9 Hz), 1.40 (m, 1H, CH), 1.28 (m, 8H, C—CH₂—C), 0.89 (t, 6H, CH₃, J=6.2 Hz). FT-IR (ATR): ν 3336 (N—H stretching), 2960, 2928, 1669 (C=O stretching), 1595, 1543, 1515, 1340, 1226, 1140, 1105, 859, 843, 685 cm⁻¹.

Aza-dye 15B: 3-(2-ethyl-hexyl)-1-(3-[4-(4-nitro-phenylazo)-phenyl]-ureido-1,4-butyl)-urea

4-Isocyanato-4′-nitroazobenzene (0.55 g, 2.07 mmol) was dissolved in THF (30 ml), and 4-(tert-butoxycarbonylamino)-1-butylamine (0.58 g, 3.11 mmol) in THF (4 ml) was added. The reaction mixture was stirred at room temperature for 30 min, after which it was partially concentrated and precipitated in pentane (100 ml). The product was filtered off, and purified by column chromatography using 1% methanol in chloroform as the eluent (R_{f}=0.3). It was redissolved in dichloromethane (3 ml), and trifluoroacetic acid (2 ml) was added to deprotect the protected amine group. The reaction mixture was stirred at room temperature overnight, and subsequently evaporated to dryness to generate the aza-amine.

Di-tert-butyl tricarbonate (0.40 1.54 mmol) was dissolved in chloroform (10 ml), and 2-ethylhexyl amine (0.19 g, 1.47 mmol) in chloroform (2 ml) was injected into the former solution. The reaction mixture was stirred for 30 min to generate a solution of 2-ethyl hexyl isocyanate. The aza-amine was dissolved in pyridine (50 ml), and was added to the solution of 2-ethyl hexyl isocyanate. The reaction mixture was stirred for 30 min at room temperature, and then evaporated to dryness. The product was purified by column chromatography, first using pure chloroform as the eluent, than chloroform-methanol mixtures with up to 10% methanol (R_{f}=0.2). The product was obtained as an orange solid (0.28 g, 27%). ¹H-NMR (10% methanol-d4 in CDCl₃): δ 8.37 (d, 2H, C2′H, J=8.1 Hz), 7.99 (d, 2H, C3′H, J=8.4 Hz), 7.93 (d, 2H, C2H, J=8.4 Hz), 7.59 (d, 2H, C3H, J=9.2 Hz), 3.26 (t, 2H, PhNHCONHCH₂), 3.15 (t, 2H, PhNHCONHCH₂CH₂CH₂CH₂), 3.07 (t, 2H, NHCONHCH₂CH), 1.54 (m, 4H, NHCH₂CH₂CH₂CH₂NH), 1.4-1.2 (m, 9H, CH+CH₂), 0.88 (t, 6H, CH₃). FT-IR (ATR): ν 3322 (N—H stretching), 2924, 2859, 1633+1623 (C=O stretching), 1584, 1552, 1523, 1343, 1226, 1140, 1106, 865, 754 cm⁻¹. UV-Vis (THF): λₘₐₓ=405 nm.

### Example 16

### Incorporation of Aza-Dyes 15A and 15B Into the Polymer Material of Example 1

Preparation of Aza-Dye Filled Films

[pTHF₁₁₀₀-U-C₄H₈-U]ₙ (ca. 2 g) and ca. 3 w/w % of aza-dye 15A or 15B were dissolved in chloroform (15 ml) and methanol (5 ml). These solutions were cast in silylated Petri-dishes (diameter 9 cm), and the solvent was allowed to evaporate slowly by placing a beaker over the dishes. After 20 h, the film was dried in vacuo at 50° C. for 5 h, and it was peeled off the Petri-dish. Both films containing either aza-dye 15A or aza-dye 15B were elastic, red and transparent. Microphase separation was not observed with optical microscopy for neither of the two films.

Washing of the Aza-Dye Filled Films With a 0.1 M Sodium Dodecylsulphate (SDS) Solution

Square pieces of 1 cm² of the prepared red films were cut and they were individually stirred in a 0.1 M sodium dodecylsulfate (SDS) solution at 60° C. for 90 minutes. This washing procedure had a remarkably different effect on the two pieces of polymer film. The film containing the aza-dye 15A that only has one urea group discoloured rapidly. After 90 minutes, it had become pale, while the washing water had an intense red colour, indicating that the aza-dye 15A is easily solubilized because it is loosely bound in the polymer material. In contrast, the piece of film containing aza-dye 15B kept its red colour. Even after prolonged washing, the washing water remained colourless, although the aza-dye 15B itself is readily soluble in the aqueous SDS-solution.

This experiment proves that the latter dye 15B is strongly anchored in the polymer material, whereas the aza-dye 15A is not, and thus can be easily washed out. The result can be explained by the fact that the aza-dye 15B and polymer [pTHF₁₁₀₀-U-C₄H₈-U]ₙ both contain bis-ureido-butylene units. This unit self-assembles, whereby the aza-dye 15B becomes strongly anchored into the polymer material.

### Example 17

### Synthesis of a RGD-Sequence Containing Peptide That Also Contains a Bis-Ureido-Butylene Unit

1-Hexyl-3-(4-isocyanato-butyl)-urea: Diisocyanatobutane (5.5 g, 39.5 mmol) was dissolved in 30 mL of dry chloroform and a solution of hexylamine (0.4 g, 3.95 mmol) in 10 mL of dry chloroform was added drop wise. The reaction was allowed to stir for 30 minutes after which the reaction mixture was filtered, the filtrate was reduced in volume and precipitated twice in hexane. A white solid was obtained in quantitative yield. FT-IR: 3325, 2955, 2930, 2860, 2264, 1614, 1571 cm⁻¹. ¹H-NMR (CDCl₃): δ=4.23 (b, 2H), 3.35 (t, 2H), 3.21 (t, 2H), 3.16 (t, 2H), 1.63, 1.49 and 1.29 (m, 12H), 0.89 (t, 3H).

The peptide: (S)-N-((S)-1-Carboxy-2-hydroxy-ethyl)-3-(2-{(S)-5-guanidino-2-[2-(6-{3-[4-(3-hexyl-ureido)-butyl]-ureido}-hexanoylamino)-acetylamino]-pentanoylamino}-acetylamino)-succinamic acid. Starting with the Wang-resin loaded with FMOC protected serine (1.5 g, 0.95 mmol), manual peptide chain assembly was carried out using DIPCDI/HOBt mediated (3.3/3.6 eq. with respect to peptide-resin) couplings in DMF. The Wang-resin loaded with FMOC protected serine was allowed to swell in DMF and the FMOC removal was achieved with 20% piperidine/DMF for 30 minutes followed by washes with DMF (3 washes at 5 minutes per wash). Three eq. of FMOC protected aminoacids were incorporated in separate syntheses; FMOC-Asp(OtBu) (1.2 g, 2.9 mmol), FMOC-Gly (0.84 g, 2.8 mmol), FMOC-Arg(PMC) (1.9 g, 2.9 mol) and FMOC-Gly (0.84 g, 2.8 mmol) were separately dissolved in DIPCDI/HOBt coupling reagents (6.5 ml) and were allowed to react at least 30 minutes with the loaded Wang-resin. Kaisertests, based on ninhydrin, showed the presence of free amine groups after each step, indicating a successful reaction (removal of FMOC or coupling of an aminoacid). The obtained product on the resin was washed with dichloromethane (2 washes at 5 minutes per wash) and with Et2O (1 wash for 5 minutes) and dried by air. FMOC removal of this FMOC-GRGDS-resin (0.63 g, 0.26 mmol) was achieved with 20% piperidine/DMF and the GRGDS-resin was washed with DMF (3 washes at 5 minutes per wash) and allowed to swell. 6-(Fmoc-amino)caproic acid (0.32 g, 0.91 mmol) dissolved in 2.1 ml DMF containing DIPCDI/HOBt (1:1:1 eq.) was allowed to react with GRGDS-resin for one hour and was then washed with DMF (3 washes at 5 minutes per wash). FMOC was again removed by 20% piperidine/DMF. Three eq. 1-hexyl-3-(4-isocyanato-butyl)-urea (0.10 g, 0.43 mmol), were added and allowed to react overnight. After filtration, the resin was washed three times with DMF and three times with DCM. The product was cleaved off the resin by 95% TFA/H20 (2 ml) at ambient conditions for six hours, filtered, precipitated in Et2O and spun down (2 minutes at 4300 RPM). The product was stirred up in Et2O and spun down three more times. The white residue was subsequently freeze dried three times from water with 10-33% acetonitrile, which resulted in a white fluffy powder. No TFA was observed anymore by ¹⁹F-NMR. LC-MS revealed one peak in the chromatogram with m/z observed mass: [M+H]+=845.5 g/mol and [M+H]2+=423.3 g/mol. Calculated mass: 844.96 g/mol.

### Example 18

### Incorporation of the Peptide of Example 17 Into the Polymer Material of Example 11

The peptide of Example 17 was incorporated into the polymer material of Example 11 and, for reference, into polycaprolactone of molecular weight 80.000. This was done by dissolving the peptide and the polymer into a THF-solution, dropcasting this solution and let the THF evaporate. In both cases, 4 mol % of peptide was used, based on the amount of bis-urea units (i.e., the bis-ureido butylene units) in the components.

Both polymer samples were incubated with water at 37° C. during 48 hrs. In the case of the pCL_{80.000} material, 49% of the peptide was extracted out of the material into the water, whereas in the case of the [pCL₂₀₀₀-U-C₄H₈-U]ₙ material of Example 11 only 26% of the peptide got extracted, implying that 74% of the peptide remained in this material. The percentages were determined using reversed phase liquid chromatography using mass spectrometry as detection (RPLC-MS).

The result shows that the peptide is anchored into the polymer material of Example 11, presumably because there is recognition between the bis-urea units present in both the polymer and the peptide component. The anchored peptide is preferably used to stimulate cell binding onto the polymer material.

### Example 19

### The Biocompatibility of the Polymer Material of Example 11

In a cell proliferation assay, the proliferation of 3T3 mouse fibroblasts on the material [pCL₂₀₀₀-U-C₄H₈-U]ₙ was compared to cell proliferation on cell culture polystyrene (PS), a known biocompatible material. Cells were seeded at a density of 1×10³ or 1×10⁴ cells/cm² in duplicate. Cell proliferation was evaluated by optical microscopy on day 1, 3, 4 and 7. In all experiments, similar behavior was observed for cells seeded on [pCL₂₀₀₀-U-C₄H₈-U]ₙ as compared to cells seeded on PS, demonstrating the biocompatibility of the material of Example 11.

In a second in vitro biocompatibility test, the cell viability of 3T3 mouse fibroblasts seeded in medium that was incubated with [pCL₂₀₀₀-U-C₄H₈-U]ₙ, UHMWPE (ultra high molecular weight polyethylene) or latex was investigated using a LDH (lactate dehydrogenase) test. Every 24 hours the medium was refreshed and all collected medium was used in a LDH activity assay. UHMWPE is known to be biocompatible while latex is not, and this was also found here: cell viabilities exceeding 90% and below 5% were found for UHMWPE and latex, respectively. The cell viability for [pCL₂₀₀₀-U-C₄H₈-U]ₙ was only approximately 50% when no prewash was applied, but after two prewashes, the cell viabilities were exceeding 90% proving the biocompatibility of the polymer material. The initial lower cell viability is attributed to the presence of small amounts of remaining solvent.

All patents, patent applications and publications referred to herein are incorporated by reference in their entirety.

What is claimed is:

1. A prosthesis device, comprising: a body at least partly formed from a segmented thermoplastic elastomer having crystallized blocks, and having at least one functional component which is able to reversibly bond to the crystallized blocks, wherein the elastomer has cartilage regenerative properties.

2. The prosthesis device of claim 1, wherein the segmented thermoplastic elastomer is a thermoplastic elastomeric polyurethane.

3. The prosthesis device of claim 1, wherein the crystallized blocks comprise at least one bis-urea moiety.

4. The prosthesis device of claim 1, wherein the at least one functional component comprises a peptide.

5. The prosthesis device of claim 4, wherein the peptide comprises at least one RGD-sequence.

6. The prosthesis device of claim 1, wherein the body provides cushioning and load distribution capabilities within a joint space, and the body is shaped such that the body fits with a femoral condyle, a tubercle, and a tibial plateau, and stays within the joint space without any separate means of attachment.

7. The prosthesis device of claim 1, further comprising a superior surface forming a concave groove channel contoured to receive a femoral condyle, and further comprising an inferior surface forming a convex surface contoured to fit on top of a tibial plateau.

8. The prosthesis device of claim 1, wherein the body is either substantially kidney shaped, substantially toroidal in shape, or substantially crescent shaped.

9. The prosthesis device of claim 1, wherein the body is attached to a tissue fixation component, selected from the group consisting of extension tabs, sutures, and mesh.

10. The prosthesis device of claim 1, wherein the body further comprises a reinforcing material selected from the group consisting of polymers and metals.

11. The prosthesis device of claim 10, wherein the reinforcing material is a foam which forms the core of the body and the elastomer skin.

12. A method for the preparation of biocompatible segmented thermoplastic elastomer, comprising:
a. dissolving an elastomer having crystallized blocks and at least one functional component which is able to reversibly bond to the crystallized blocks into a solvent to form a solution;
b. mixing the solution; and,
c. at least partly evaporating the solvent to yield a biocompatible segmented thermoplastic elastomer having cartilage regenerative properties.

13. A biocompatible segmented thermoplastic elastomer, comprising: crystallized blocks, and at least one functional component which is able to reversibly bond to the crystallized blocks, wherein the elastomer has cartilage regenerative properties and can be used in a prosthesis device able to grow into cartilage.

14. A method for inserting a prosthesis device into a joint space, comprising:
a. providing a prosthesis device comprising a body at least partly formed from a segmented thermoplastic elastomer having crystallized blocks, and having at least one functional component which is able to reversibly bond to the crystallized blocks, wherein the elastomer has cartilage regenerative properties;
b. making an incision in the tissue surrounding the joint space of a knee;
c. inserting the prosthesis device into the joint space of the knee; and
d. closing the incision.

15. A method for inserting a prosthesis device into a bone structure, comprising:
a. providing a prosthesis device comprising a body at least partly formed from a segmented thermoplastic elastomer having crystallized blocks, and having at least one functional component which is able to reversibly bond to the crystallized blocks, wherein the elastomer has cartilage regenerative properties;
b. making an incision in the tissue surrounding the bone structure;
c. boring a hole into the bone structure;
d. inserting the prosthesis device into the hole; and
e. closing the incision.
